(19) United States
(12) Patent Application Publication
(10) Pub. No.: US 2011/0207114 A1
(43) Pub. Date: Aug. 25, 2011

(54) Amplified Fluorescence Polymers and Sensor Thereof

(75) Inventors: Anil Kumar, Maharashtra (IN); Jasmine Sinha, Bihar (IN); Phani Kumar Pullela, Karnataka (IN)

(73) Assignees: INDIAN INSTITUTE OF TECHNOLOGY MUMBAI, Mumbai, Maharashtra (IN); BIGTEC PRIVATE LIMITED, Bangalore, Karnataka (IN)

(21) Appl. No.: 13/126,204

(22) PCT Filed: Oct. 29, 2009
(86) PCT No.: PCT/IB09/07260

§ 371 Date: Apr. 27, 2011

(30) Foreign Application Priority Data
Oct. 29, 2008 (IN) 02319/MUM/2008

(51) Int. Cl. C12Q 1/00; G01N 33/00; G01N 33/50; G01N 33/92; C08G 75/12; C08F 228/06; C08F 238/00

(52) U.S. Cl. 435/4; 436/104; 436/116; 436/86; 436/71; 436/94; 528/380; 526/256

(57) ABSTRACT

The present disclosure is related to amplified fluorescence polymers (AFPs) with pendant functional groups, their derivatives and their synthesis. The amplified fluorescence polymers can be used in various biological and chemical sensors.

### FIELD OF THE DISCLOSURE

The disclosure relates to amplified fluorescence polymers (AFPs) with pendant functional groups, their derivatives, synthesis and applications in sensors.

### BACKGROUND AND PRIOR ART

Amplified fluorescence polymers belong to an important class of polymers which fluoresce in solid state and hence make them a potential candidate for sensing application. A classical example of these polymers is the sensor developed by Prof. Swager's groups and U.S. Patent publication No. US20070081921 describes these polymers in detail.

The inherent disadvantages of the above mentioned synthetic strategy for the design of AFPs are the inability of the final polymers to be modified further. In other words, the final polymers do not exhibit pendant functionality which is easy to modify to incorporate various chemical and biological receptors after the polymerization. This means that all the modifications have to be carried out at the monomer stage followed by polymerization to get the final AFPs. This limits the possible applications derived from the AFP, as most of the antibodies, proteins, nucleic acid derivitizations on these polymers can be performed only after post polymerization. Usually, polymerization of AFPs is through harsh palladium catalysis, which cannot tolerate any acid, alcohol, amine, carbonyl, aldehyde, and amide functional groups. Almost all biological systems contain, one of the above stated functional groups and hence, post polymerization pendant groups will enhance the scope AFPs by many fold.

It would have been of tremendous advantage if it is possible to design AFPs with pendant functional groups which can be modified after the polymerization without protection-deprotection strategy. In this direction, we report the syntheses and characterization of a series of AFPs with pendant propargyl functional group which can be modified using “click” chemistry after the polymer has been formed and purified. The demonstrated applications of the pendant AFPs, show a great promise for development of future medical devices, chips, sensors, dipsticks, remote control systems for agricultural, clinical and animal husbandry applications, field devices, and on-board sensors.

### OBJECTS OF DISCLOSURE

One of the objects of present disclosure is to develop amplified fluorescence polymers with pendant functional groups.

Another object of the disclosure is to use the amplified fluorescent polymers with pendant functional groups in sensors for detecting various analytes.

### STATEMENT OF DISCLOSURE

The present disclosure is in relation to a compound of formula I,

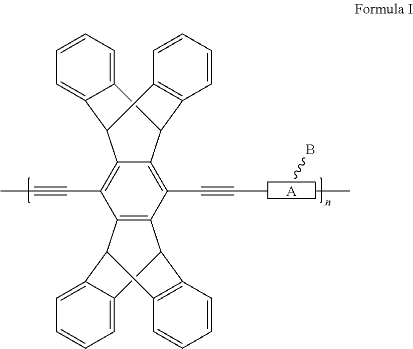

Wherein

B is selected from a group comprising but not limiting to —O—R—CCH, —O—CCH, —O—RCOOH, —O—RCHO, —O—RNH₂ and —O—R—N₃; R is selected from a group comprising but not limiting to linear or branched aliphatic C₁-C₂₀ alkyl chain, cycloaliphatic C₃-C₂₀ compound and aromatic C₅-C₂₀ compound optionally substituted with suitable functional group selected from a group comprising OH, N₃, R₁CCH, NH₂, NO₂, CHO, COOH and CN; A is selected from a group comprising aryl, heteroaryl, cycloalkyl, heterocycloalkyl groups optionally substituted with suitable functional groups selected from a group comprising OH, N₃, R₁CCH, NH₂, NO₂, CHO, COOH and CN; R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and n ranges from 1 to about 15,000; a compound of formula II,

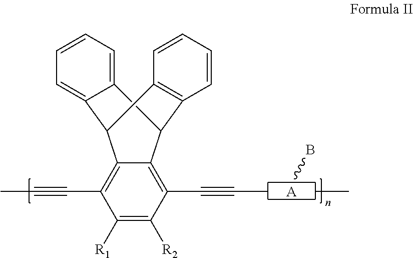

Wherein B is selected from a group comprising but not limiting to —O—R—CCH, —O—CCH, —O—RCOOH, —O—RCHO, —O—RNH₂ and —O—R—N₃; R is selected from a group comprising but not limiting to linear or branched aliphatic C₁-C₂₀ alkyl chain, cycloaliphatic C₃-C₂₀ compound and aromatic C₅-C₂₀ compound; optionally substituted with suitable functional group selected from a group comprising OH, N₃, R₁CCH, NH₂, NO₂, CHO, COOH and CN; A is selected from a group comprising aryl, heteroaryl, cycloalkyl, heterocycloalkyl groups; optionally substituted with suitable functional groups selected from a group comprising OH, N₃, R₁CCH, NH₂, NO₂, CHO, COOH and CN; R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and n ranges from 1 to about 15,000; a compound of formula III,

—P-Q- Formula III

Wherein,

P is amplified fluorescent polymer of formula I of claim 1 or formula II of claim 2; and

Q is selected from a group comprising

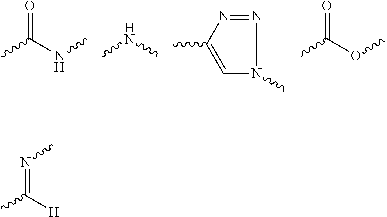

a sensor for detecting an analyte comprising compound of formula III;

—P-Q- Formula III

P is amplified fluorescent polymer of formula I of claim 1 or formula II of claim 2; and

Q is selected from a group comprising

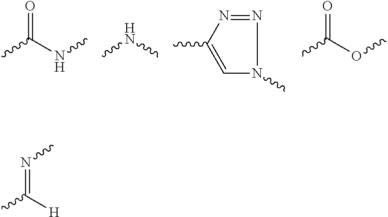

a method of detection of an analyte in a sample, said method comprising steps of; contacting compound of formula III or sample comprising compound of formula III with sample in presence of a solvent; and determining change in fluorescence of the reacted compound of formula III to detect the analyte.

### BRIEF DESCRIPTION OF ACCOMPANYING DRAWING

FIG. 1: shows the change in the fluorescence of the amplified fluorescent polymer during the detection of electrophile.

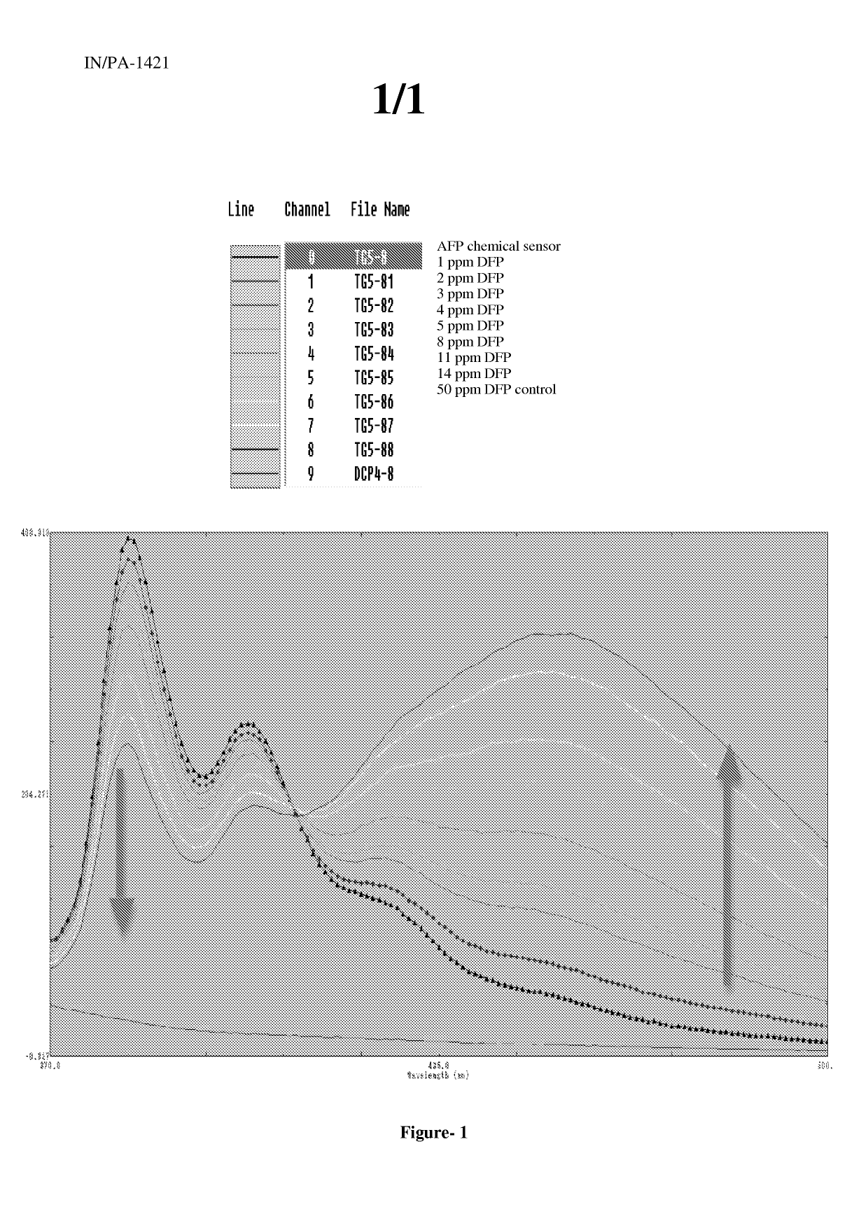

### DETAILED DESCRIPTION OF DISCLOSURE

The disclosure is in relation to a compound of formula I,

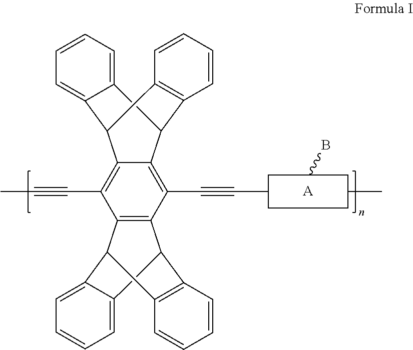

Wherein

B is selected from a group comprising but not limiting to —O—R—CCH, —O—CCH, —O—RCOOH, —O—RCHO, —O—RNH₂ and —O—R—N₃;

R is selected from a group comprising but not limiting to linear or branched aliphatic C₁-C₂₀ alkyl chain, cycloaliphatic C₃-C₂₀ compound and aromatic C₅-C₂₀ compound optionally substituted with suitable functional group selected from a group comprising OH, N₃, R₁CCH, NH₂, NO₂, CHO, COOH and CN;

A is selected from a group comprising aryl, heteroaryl, cycloalkyl, heterocycloalkyl groups optionally substituted with suitable functional groups selected from a group comprising OH, N₃, R₁CCH, NH₂, NO₂, CHO, COOH and CN;

R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and

n ranges from 1 to about 15,000.

The disclosure is also in relation to a compound of formula II,

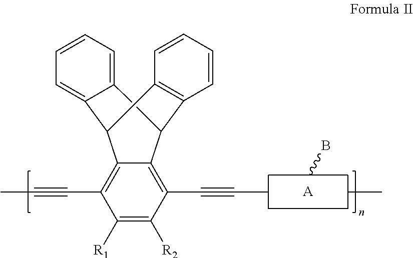

Wherein

B is selected from a group comprising but not limiting to —O—R—CCH, —O—CCH, —O—RCOOH, —O—RCHO, —O—RNH₂ and —O—R—N₃;

R is selected from a group comprising but not limiting to linear or branched aliphatic C₁-C₂₀ alkyl chain, cycloaliphatic C₃-C₂₀ compound and aromatic C₅-C₂₀ compound; optionally substituted with suitable functional group selected from a group comprising OH, N₃, R₁CCH, NH₂, NO₂, CHO, COOH and CN;

A is selected from a group comprising aryl, heteroaryl, cycloalkyl, heterocycloalkyl groups; optionally substituted with suitable functional groups selected from a group comprising OH, N₃, R₁CCH, NH₂, NO₂, CHO, COOH and CN;

R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and

n ranges from 1 to about 15,000.

In another embodiment of the present disclosure, A is selected from a group comprising

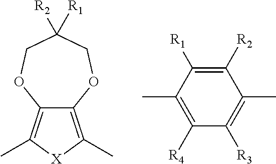

wherein

R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN;

X is selected from a group comprising but not limiting to H, O, S and NR;

R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN;

R₂ is selected from a group comprising but not limiting to the B, linear or branched aliphatic C₁-C₂₀ alkyl chain, cycloaliphatic C₃-C₂₀ compound, and aromatic C₅-C₂₀ compound comprising B; optionally substituted with functional groups comprising OH, N₃, R₁CCH, NH₂, NO₂, CHO, COOH and CN;

R₃ is selected from a group comprising but not limiting to the B, linear or branched aliphatic C₁-C₂₀ alkyl chain, cycloaliphatic C₃-C₂₀, and aromatic C₅-C₂₀ compound comprising the B; optionally substituted with functional groups comprising OH, N₃, R₁CCH, NH₂, NO₂, CHO, COOH and CN; and

R₄ is selected from a group comprising but not limiting to the B, linear or branched, aliphatic C₁-C₂₀ alkyl chain, cycloaliphatic C₃-C₂₀ compound, aromatic C₅-C₂₀ compound comprising the B; optionally substituted with functional groups comprising OH, N₃, R₁CCH, NH₂, NO₂, CHO, COOH and CN.

The disclosure is also in relation to a compound of formula III,

—P-Q- Formula III

Wherein,

P is amplified fluorescent polymer of formula I or formula II 2; and

Q is selected from a group comprising

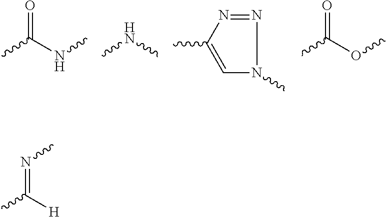

The disclosure is also in relation top a sensor for detecting an analyte comprising compound of formula III;

—P-Q- Formula III

P is amplified fluorescent polymer of formula I or formula II; and

Q is selected from a group comprising

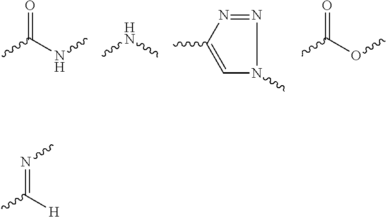

A sensor for detecting an analyte comprising compound of formula III;

—P-Q- Formula III

P is amplified fluorescent polymer of formula I of claim 1 or formula II of claim 2; and

Q is selected from a group comprising

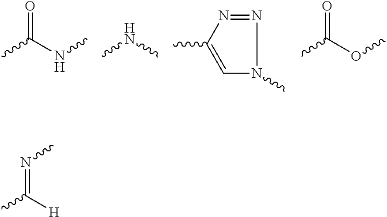

In another embodiment of the disclosure, the sensor comprises buffer, alcohol and detector.

In still another embodiment of the disclosure, the buffer is selected from a group comprising sodium acetate, potassium acetate, piperidine, ethanolamine, pyridine, pyrizine, tristriphine, MOPS and MES

In still another embodiment of the disclosure, the alcohol is selected but not limiting to a group comprising methyl alcohol, ethyl alcohol, isopropyl alcohol, n-butyl alcohol and t-butyl alcohol.

In still another embodiment of the disclosure, the detector is fluorescence detector. The present disclosure is also in relation to a method of detection of an analyte in a sample, said method comprising steps of;
a. contacting compound of formula III or sample comprising compound of formula III with sample in presence of a solvent; and
b. determining change in fluorescence of the reacted compound of formula III to detect the analyte.

In still another embodiment of the disclosure, concentration of the analyte is detected.

In still another embodiment of the disclosure, the detection of the analyte is with the help of sensor.

In still another embodiment of the disclosure, the solvent is selected from a group not limiting to dichloromethane, chloroform, water, aqueous burrer with pH ranging from about 2 to about 12, preferably ranging from about 5 to about 9, hexane, ethyl acetate, acetone, toluene, xylene, dimethyl sulphoxide, N,N-dimethyl formamide, acetic acid, formic acid, H₂SO₄, HCl and HNO₃

In still another embodiment of the disclosure, the analyte is a solid, liquid, gas and mixture thereof.

In still another embodiment of the disclosure, the analyte is selected from a group comprising chemical analyte and biochemical analyte.

In still another embodiment of the disclosure, the chemical analyte is selected from a group comprising but not limiting to samples obtained from environmental studies and industrial effluents.

In still another embodiment of the disclosure, the sample is selected from a group comprising organo phosphates, nitro compounds, electrophiles and nucloephiles.

In still another embodiment of the disclosure, the biochemical analyte is extracted from a biological sample selected from a group comprising serum, blood, plasma, saliva, urine, feces, seminal plasma, sweat, liquor, amniotic fluid, tissue homogenate and ascites.

In still another embodiment of the disclosure, the biochemical analyte is selected from a group comprising but not limiting to peptides, proteins, antibodies, hormone, lecithin, enzymes, DNA and RNA.

a method of detection of an analyte in a sample, said method comprising steps of; contacting compound of formula III or sample comprinsing compound of formula III with sample in presence of a solvent; and determining change in fluorescence of the reacted compound of formula III to detect the analyte;

In an embodiment of the disclosure, the amplified fluorescent polymers (AFP) described and synthesized herein have advantages over prior arts in terms of:
1. Ease of the syntheses of AFPs with pendant functional group capable of undergoing “Click” chemistry after the polymerization.
2. Possibility of post polymerization functionalization to incorporate chemical and biological receptors in order to design and fabricate chemical and biochemical sensors.

### Applications of the Polymer:

In yet another embodiment of the disclosure, the polymer of formula I and II can be used in following applications

### 1. For Detection of Electrophile

The below molecule is synthesized to detect electrophiles (AFP chemical sensor). The t-butyldimethylsilyl functional group reacts with any electrophile and forms a positively charged cyclic product, which forms a fluorescence resonance energy transfer (FRET) paid with the polymer. When the polymer is excited, the fluorescence from polymer is transferred to the newly formed fluorescent if strong electrophiles are present and if no electrophiles are present, the polymer gives the emission.

Following polymer was dissolved in dichloromethane and a strong electrophile (diisopropyl ethyl fluoro phosphate (DFP)) was exposed at 1-14 ppm. The polymer was excited at 360 nm and it has emission maximum at 383 nm and 404 nm. The cyclized product (formed by a bond between naphthyl ring and pyridine ring due to intramolecular cyclization) has absorption maximum at 400 nm and gives fluorescence emission at 450 nm. The cyclized product formation initiates a FRET between the polymer and cyclized produce with which the excitation at 360 nm results in increase of emission at 450 nm and reduction of emission of polymer at 383 and 404 nm. It was observed that, only strong electrolytes like DFP give the 450 nm peak and weak electrolytes like methyl parathion and ethyl paraoxon does not give any signal indicating it is a specific sensor for strong electrolytes. Strong electrolytes are concentrated acids like HNO₃, H₂SO₄, HCl, thionyl chloride etc are not commonly present in atmosphere.

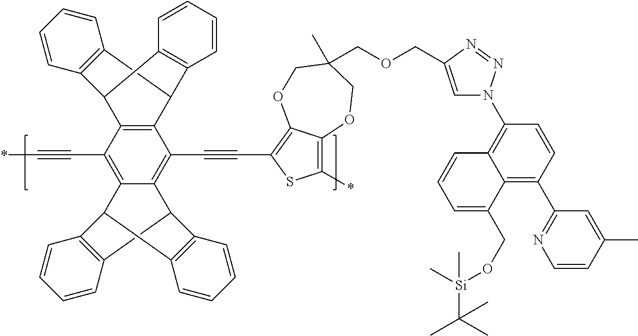

The AFP polymer was dissolved in dichloromethane and a strong electrophile (diisopropyl ethyl fluoro phosphate (DFP)) was exposed at 1-14 ppm. The polymer was excited at 360 nm and it has emission maximum at 383 nm and 404 nm. When the polymer was exposed with DFP, there is a gradual decrease of fluorescence intensity at 383 nm and 404 nm, and a new peak at 450 nm, indicating formation of a new fluorescence species, which absorbs in the polymer emission region (383 & 404 nm) and emits at 450 nm.

### 2. For Detection of any Industrial Effluent

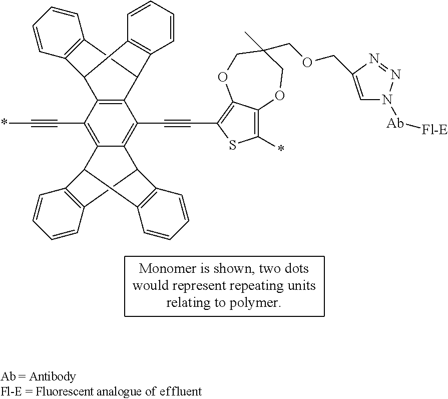

The target effluent will be competing with the antibody bound fluorescent compound and the displacement of fluorescent analogue of target effluent compound is monitored by FRET.

### 3. For Detection of Specific Contaminants in Milk.

Adulteration of milk is a serious concern and contaminants need to be identified at very low concentrations.

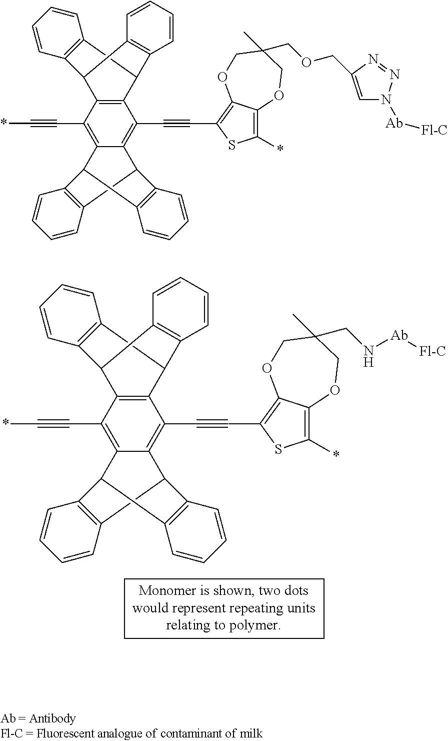

### Detection of Melamine in Milk

Immobilise melamine specific monoclonal or polyclonal antibody onto the polymer via amide bond

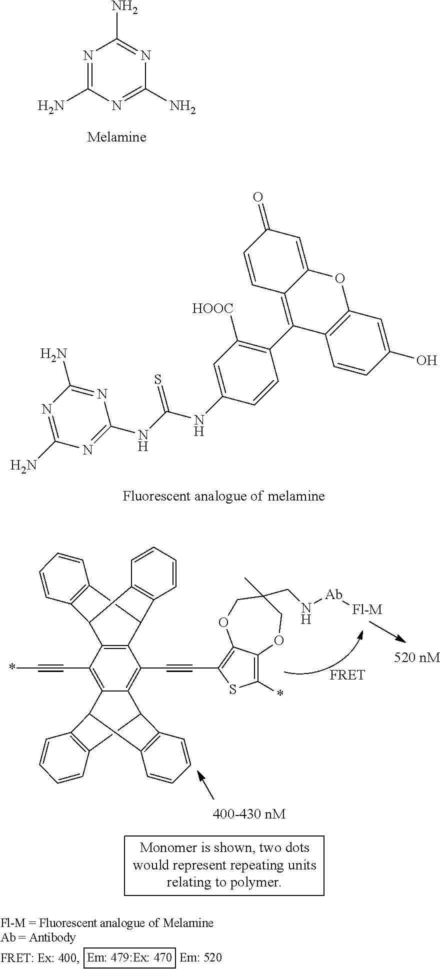

### 4. Quantization of Protein Related to Kidney Injury.

The biomarkers for acute and chronic kidney injury are Cystatin C and NGAL. Both these proteins are difficult to assay and methods for their assay are needed. The present disclosure provides a method for the same given in the steps below.

Step 1: To the polymer containing decanoic acid, NGAL sample is added and incubated protein bound polymer is taken to step 2.

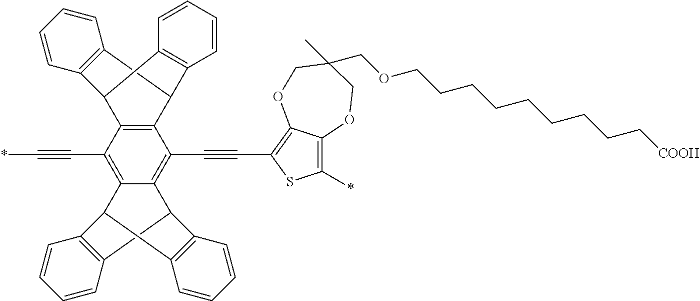

Polymer to detect and quantitate NGAL

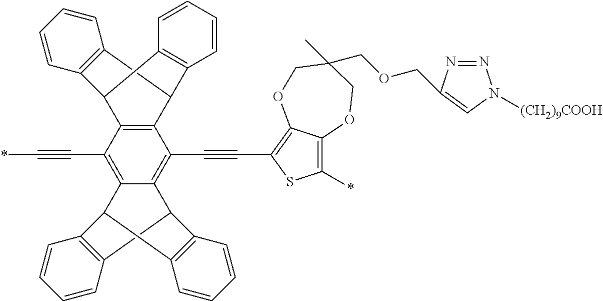

Step 2: Here the NGAL specific antibody labeled with a fluorescent molecule is added and incubated.

The fluorescent molecule on antibody and the fluorescence form polymer form a FRET pair. There is an increase in FRET signal corresponding to concentration of NGAL.

### 5. Quantification of Proteins Related to Cardiovascular Disease and Cancer

There are protein biomarkers which can predict more accurately about Heart attack and cancer. It is important to target these protein biomarkers for prediction of cardiovascular diseases and cancer.

Below approach will be used to target any protein biomarker related to cardiovascular diseases and cancer.

### Genes Responsible for Cancer and Cardiovascular Disease

Genes of interest will be amplified by RT-PCR The probes are tagged with single fluorescent probe and attached to the polymer by Click chemistry

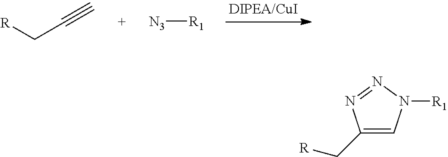

Acid and amine condensation

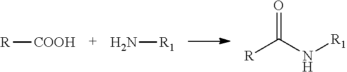

Aldehyde and amine: Schiff's base preparation

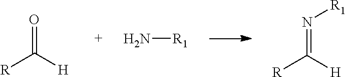

The polymer and fluorescent probe on the probe term a FRET pair and as the PCR cycles go on, the FRET signal will decrease.

### 6. Identification of Infectious Disease Based on PCR.

RT-PCR revolutionized the disease diagnosis the inherent drawback of RT-PCR is reliance in fluorescence probes labeled on both sides of the sequence (TAQMAN chemistry). TAQMAN probes are selective but at same point multiplexing is impossible. Secondly, the background fluorescence complicates the diagnosis.

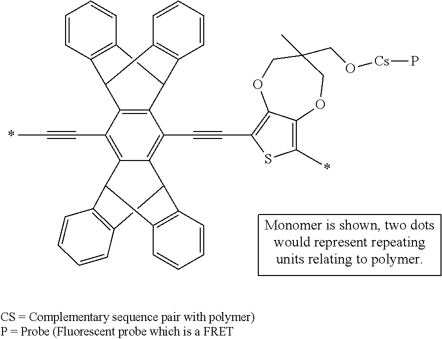

Whenever PCR cycle goes one probe is cut and corresponding FRET is lost, as the PCR cycles increase, there will be decrease in FRET signal, which indicates positive discrete signal.

### 7. Target DNA Amplification for any Human Disease or Health Condition.

Method is as described in previous method using PCR

### 8. For Simultaneous Identification of Multiple Biomarkers for Organ Specific Diseases

The polymer back bone is made in such a way that there are multiple ligands attached to polymer when any sample, saliva, serum or urine was added multiple proteins bind to corresponding ligands this way we develop antibodies tagged to fluorescent compounds for these proteins and each form an individual FRET pair with same excitation, preferably at the excitation wavelength of the polymer.

The polymer can be used in medical devices, chips, sensors, dipsticks, remote control systems for agricultural, clinical and animal husbandry applications, field devices, and on-board sensors.

### 9. For Detection of Pesticide:

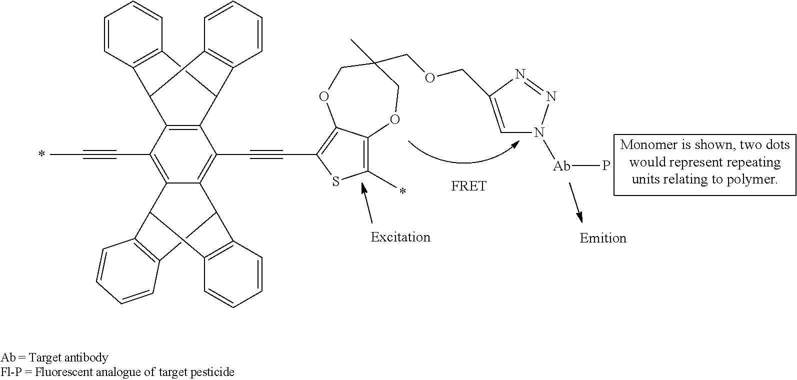

The target pesticide will be competing with the antibody based fluorescent compound and the displacement of fluorescent analogue of target is monitored by FRET.

### Sensor:

A sensor is made by coating the polymer on a chip or a film or a strip or any other solid surface, which will come in contact with analyte

The coating of polymer on the solid surface is done by dissolving polymer in a solvent and drying the polymer on the surface of the solid. The coated polymer could be formula I or Formula II or Formula III compound. If the coated polymer is Formula I or Formula II, then it is derivitized with an appropriate compound with “Q” and then treated with the analyte. The formula III compound will be directly exposed to the analyte.

The disclosure is further elaborated with the help of following examples. However, these examples should not be construed to limit the scope of disclosure.

### EXAMPLES

### I. Preparation of Polymer of the Representative Structures in Table 1

### A. Synthesis of 6,8-Dibromo-3,3-Dibenzyl-3,4-dihydro-2H-thieno[3,4-b][1,4]dioxepine

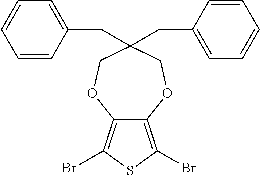

3,3-Dibenzyl-3,4-dihydro-2H-thieno[3,4-b][1,4]dioxepine (0.05 g, 0.14 mmol) was taken with chloroform and added to it bromine (0.049 g, 0.31 mmol) with temperature maintained at 10-15° C. On completion of bromine addition, reaction mixture was continued for stirring at room temperature for 3 h. After the completion of the reaction, the reaction mixture was poured into the solution of sodium hydroxide to quench excess of bromine and washed with water several times. On neutralization pale white solid was obtained. Yield: 92%; ¹H NMR (CDCl₃, 400 MHz, δ ppm): 2.82 (s, 4H), 3.91 (s, 4H) 7.15 (m, 2H), 7.27 (m, 4H), 7.33 (m, 4H); ¹³C NMR (CDCl₃, 100 MHz): 39.6, 45.2, 77.9, 2.6, 126.6, 128.3, 130.6, 136.0, 147.4; MALDI-TOF: 485.85 (M⁺+Na); Anal. Calcd for C₂₁H₁₈Br₂O₂S (494): C, 51.29; H, 3.35. Found: C, 51.03; H, 3.67.

### B. Synthesis of 3,3-Dibenzyl-6,8-bis-trimethylsilanylethynyl-3,4-dihydro-2H-thieno[3,4-b][1,4]dioxepine

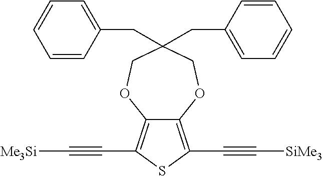

6,8-Dibromo-3,3-Dibenzyl-3,4-dihydro-2H-thieno[3,4-b][1,4]dioxepine (0.1 g, 0.2 mmol) and trimethylsilylacetylene (0.05 g, 0.54 mmol) was taken in diisopropylamine (2 mL) and added to it CuI (0.77 mg, 0.004 mmol) followed by the addition of Pd(PPh₃)₄ (0.016 g, 0.01 mmol). The reaction mixture was refluxed overnight. The reaction mixture was then subjected to CHCl₃/H₂O workup. The combined organic layer was then washed with NH₄Cl water solution, and then dried over Na₂SO₄. The solvent was then evaporated to obtain a brown liquid which was then column purified to get a light yellow liquid. Yield: 98%; ¹H NMR (CDCl₃, 400 MHz, δ ppm): 0.24 (s, 18H), 2.85 (s, 4H), 3.92 (s, 2H), 7.2-7.3 (m, 10H); ¹³C NMR (CDCl₃, 100 MHz): −0.122, 39.503, 45.230, 77.862, 94.776, 102.654, 104.911, 126.638, 128.293, 130.664, 136.269, 151.461; HR MS (C₃₁H₃₆O₂SSi₂): calcd 529.2053. found 529.2040.

### C. Synthesis of 3,3-Dibenzyl-6,8-diethynlyl-3,4-dihydro-2H-thieno[3,4-b][1,4]dioxepine

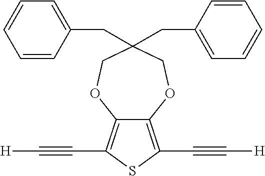

3,3-Dibenzyl-6,8-bis-trimethylsilanylethynyl-3,4-dihydro-2H-thieno[3,4b][1,4]dioxepine (0.125 g, 0.23 mmol) was the dissolved in MeOH/THF and added to it K₂CO₃ (0.071 g, 0.52 mmol) and kept for stirring at room temperature for 5 h. The resulting brown liquid was then subjected to CHCl₃/H₂O workup. The combined organic layer was then washed and dried over Na₂SO₄. The solvent was then evaporated to obtain a brown liquid which was then column purified to get a pale yellow liquid. Yield: 75%; ¹H NMR (CDCl₃, 400 MHz, δ ppm): 2.85 (s, 4H), 3.69 (s, 2H), 3.92 (s, 2H), 7.2-7.3 (m, 10H). ¹³C NMR (CDCl₃, 100 MHz): 39.564, 45.245, 76.361, 78.014, 84.611, 104.149, 126.714, 128.338, 130.626, 136.048, 152.162; ES MS: 385.09 [M+1]⁺; HR MS (C₂₅H₂₀O₂S): calcd 385.1262. found 385.1261.

### D. Synthesis of 6,8-Dibromo-3,3-Dihexyl-3,4-dihydro-2H-thieno[3,4-b][1,4]dioxepine

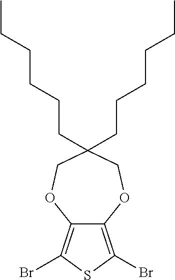

3,3-Dihexyl-3,4-dihydro-2H-thieno[3,4-b][1,4]dioxepine (0.8 g, 2.4 mmol) was taken with chloroform and added to it NBS (0.96 g, 5.4 mmol), reaction mixture was continued for stirring at room temperature for 12 h. After the completion of the reaction, the reaction mixture was poured into water. The combined organic layer was then washed with water and then dried over Na₂SO₄. The solvent was then evaporated to obtain a brown liquid which was then column purified to get a colorless liquid. Yield: 98%; ¹H NMR (CDCl₃, 400 MHz, δ ppm): ¹H NMR (CDCl₃, 300 MHz): δ 0.86 (t, J=6.8 Hz, 6H), 1.28-1.39 (m, 20H), 3.84 (s, 4H); ¹³C NMR (CDCl₃, 100 MHz): 14.032, 22.596, 22.626, 30.001, 31.564, 31.663, 43.941, 77.969, 90.590, 147.098.

### E. Synthesis of 3,3-Dihexyl-6,8-bis-trimethylsilanylethynyl-3,4-dihydro-2H-thieno[3,4-b][1,4]dioxepine

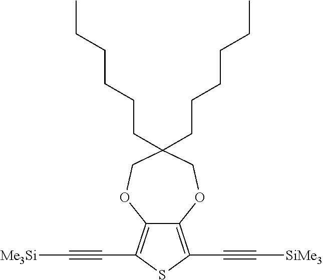

6,8-Dibromo-3,3-Dihexyl-3,4-dihydro-2H-thieno[3,4-b][1,4]dioxepine (0.5 g, 1.03 mmol) and trimethylsilylacetylene (0.26 g, 2.7 mmol) was taken in diisopropylamine (8 mL) and added to it CuI (3.9 mg, 0.002 mmol) followed by the addition of Pd(PPh₃)₄ (0.059 g, 0.005 mmol). The reaction mixture was refluxed overnight. The reaction mixture was then subjected to CHCl₃/H₂O workup. The combined organic layer was then washed with NH₄Cl water solution, and then dried over Na₂SO₄. The solvent was then evaporated to obtain a brown liquid which was then column purified to get a light yellow liquid. Yield: 93%; ¹H NMR (CDCl₃, 400 MHz, δ ppm): 0.233 (s, 18H), 0.88 (t, J=6.4 Hz, 6H), 1.2-14 (m, 20H), 3.91 (s, 4H); ¹³C NMR (CDCl₃, 100 MHz): −0.107, 14.055, 22.672, 30.024, 31.694, 31.724, 43.758, 77.885, 94.929, 102.318, 103.470, 151.247; ES MS: 517.29 [M+1]⁺; HR MS (C₂₉H₄₈O₂Si₂S): calcd 517.2992. found 517.2970.

### F. Synthesis of 6,8-diethynyl-3,3-dihexyl-3,4-dihydro-2H-thieno[3,4-b][1,4]dioxepine

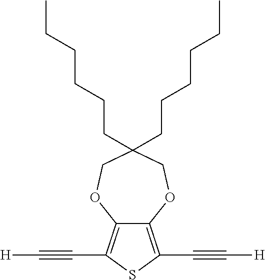

3,3-Dihexyl-6,8-bis-trimethylsilanylethynyl-3,4-dihydro-2H-thieno[3,4-b][1,4]dioxepine (0.5 g, 0.96 mmol) was the dissolved in MeOH/THE and added to it K₂CO₃ (0.29 g, 2.1 mmol) and kept for stirring at room temperature for 5 h. The resulting brown liquid was then subjected to CHCl₃/H₂O workup. The combined organic layer was then washed and dried over Na₂SO₄. The solvent was then evaporated to obtain a brown liquid which was then column purified to get a pale yellow liquid. Yield: 82%; ¹H NMR (CDCl₃, 400 MHz, δ ppm): 0.88 (t, J=3.05 Hz, 6H), 1.2-1.5 (m, 20H), 3.46 (s, 2H), 3.97 (s, 4H); ¹³C NMR (CDCl₃, 100 MHz): 14.04, 22.611, 22.665, 29.993, 31.694, 31.732, 43.812, 74.476, 77.816, 84.42, 102.296, 151.857; ES MS: 373.21[M+1]⁺; HR MS (C₂₃H₃₂O₂S): calcd 373.2201. found 373.2197.

### G. 3-Methyl-3-prop-2-ynyloxymethyl-3,4-dihydro-2H-thieno[3,4-b][1,4]dioxepine (Pro-DOT-propargyl)

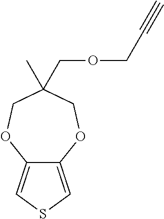

To a suspension of 0.75 g (31.2 mmol) of NaH (60% in mineral oil) in 5 mL anhydrous THF, a solution of 2.5 g (12.5 mmol) of Pro-DOT-OH in 5 mL anhydrous THF was added dropwise under argon atmosphere. The resulting mixture was stirred for half an hour followed by the addition of DABCO (lcrystal) in catalytic amount. Then to the mixture 3.15 g (15 mmol) of propargyl tosylate was added dropwise. The mixture was stirred for 48 h under argon atmosphere. The reaction mixture was poured then into 100 mL water and extracted 3-4 times from 40 mL ethyl acetate. The combined organic layer was dried over Na₂SO₄ and concentrated under reduced pressure to get dark brown viscous oil. The crude product was then purified by silica gel column chromatography eluting with petroleum ether, ethyl acetate mixture to get pale yellow liquid. Yield: 50.4%; ¹H NMR (CDCl₃, 400 MHz, δ ppm): 0.99 (s, 3H), 2.44 (t, 1H, J=2.4 Hz), 3.59 (s, 2H), 3.72 (d, 2H, J=11.9 Hz), 4.02 (d, 2H, J=11.9 Hz), 4.17 (dd, 2H, J=2.4 Hz), 6.48 (s, 1H); ¹³C NMR (CDCl₃, 100 MHz): 17.45, 43.26, 58.89, 72.64, 74.71, 76.70, 105.71, 149.89; IR (cm⁻¹): 32.88, 2962, 28.56, 2117, 1634, 1568, 1486, 1450, 1328, 1267, 1226, 1186, 1134, 1098, 1032, 849, 784, 729, 626; HRMS: calcd for C₁₂H₁₄O₃S (M+H)⁺, 239.0742. found, 239.0737.

### H. (6,8-Dibromo-3-methyl-3-prop-2-ynyloxymethyl-3,4-dihydro-2H-thieno[3,4-b][1,4]dioxepine

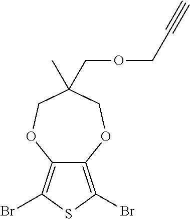

To a solution of 0.1 g (0.42 mmol) of Pro-DOT-propargyl in 1 mL of chloroform was added 0.4 g (2.2 mmol) of NBS. The solution was kept for stirring for 5 h. The resulting mixture was poured into 10 mL of water and extracted twice from 20 mL of ethyl acetate. The combined organic layer was dried over Na₂SO₄ and concentrated under reduced pressure to yield yellow solid. ¹H NMR (CDCl₃, 400 MHz, δ ppm): 0.99 (s, 3H), 2.44 (t, 1H, J=2.4 Hz), 3.59 (s, 2H), 3.72 (d, 2H, J=11.9 Hz), 4.02 (d, 2H, J=11.9 Hz), 4.17 (dd, 2H, J=2.4 Hz); ¹³C NMR (CDCl₃, 100 MHz): 17.23, 43.27, 58.70, 72.27, 74.65, 79.38, 91.52, 147.06; HRMS: calcd for C₁₂H₁₂Br₂O₃S (M+H)⁺, 394.895. found, 394.896.

### Example 1

### Polymerization of diethynylpentiptycene and 6,8-Dibromo-3,3-Dibenzyl-3,4-dihydro-2H-thieno[3,4-b][1,4]dioxepine (P1)

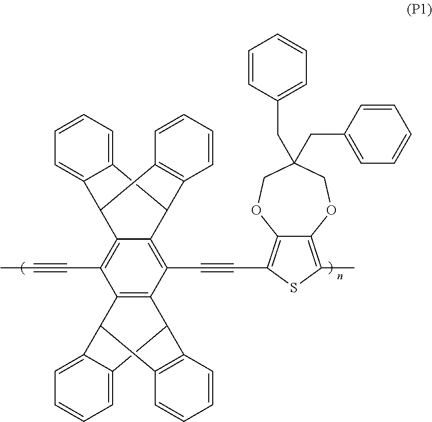

Under an atmosphere of argon, diisopropylamine/toluene (2:3, 5 mL) solvent was added to compound Diethynylpentiptycene (0.04 g, 0.084 mmol) and 6,8-Dibromo-3,3-Dibenzyl-3,4-dihydro-2H-thieno[3,4-b][1,4]dioxepine (0.041 g, 0.084 mmol) along with CuI (0.011 g, 0.053 mmol) and Pd(PPh₃)₄ (0.01 g, 8.6 mol). The reaction mixture was then kept for reflux for 5 days at a constant temperature of 65° C. The reaction mixture was then subjected to CHCl₃/H₂O workup. The combined organic layer was then washed with NH₄Cl water solution, and then dried over Na₂SO₄. The solvent was then evaporated to obtain a yellow solid, and was then reprecipitated in methanol thrice. Yield: 80%; ¹H NMR (CDCl₃, 400 MHz, δ ppm): 3.06 (br, 4H), 4.35 (br, 4H), 6.94 (br, 4H), 7.2-7.3 (br, 26H).

The absorption and emission spectra of the polymer was recorded in chloroform. The absorbance spectra shows peak at 407 nm. The emission peak was observed to be at 468 nm. M_{w} of the polymer was found to be approximately 1,130,00.

### Example 2

### Polymerization of diethynylpentiptycene and 6,8-Dibromo-3,3-Dihexyl-3,4-dihydro-2H-thieno[3,4-b][1,4]dioxepine (P2)

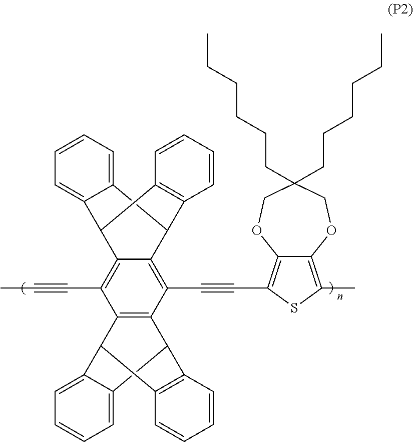

Under an atmosphere of argon, diisopropylamine/toluene (2:3, 5 mL) solvent was added to compound Diethynylpentiptycene (0.04 g, 0.084 mmol) and 6,8-Dibromo-3,3-Dihexyl-3,4-dihydro-2H-thieno[3,4-b][1,4]dioxepine (0.04 g, 0.084 mmol) along with CuI (0.011 g, 0.053 mmol) and Pd(PPh₃)₄ (0.01 g, 8.6 mol). The reaction mixture was then kept for reflux for 5 days at a constant temperature of 65° C. The reaction mixture was then subjected to CHCl₃/H₂O workup. The combined organic layer was then washed with NH₄Cl water solution, and then dried over Na₂SO₄. The solvent was then evaporated to obtain a yellow solid, and was then reprecipitated in methanol thrice. Yield: 72%; ¹H NMR (CDCl₃, 400 MHz, δ ppm): 0.86 (b, 6H), 1.2-1.5 (b, 20H), 3.06 (br, 4H), 3.97 (b, 4H), 4.35 (br, 4H), 6.94 (br, 4H), 7.2-7.3 (br, 26H).

The absorption and emission spectra of the polymer was recorded in chloroform. The absorbance spectra shows peak at 429 nm. The emission peak was observed to be at 473 nm. M_{w} of the polymer was found to be approximately 1,180,00.

### Example 3

### Polymerization of diethynylpentiptycene and (6,8-Dibromo-3-methyl-3-prop-2-ynyloxymethyl-3,4-dihydro-2H-thieno[3,4-b][1,4]dioxepine (P5)

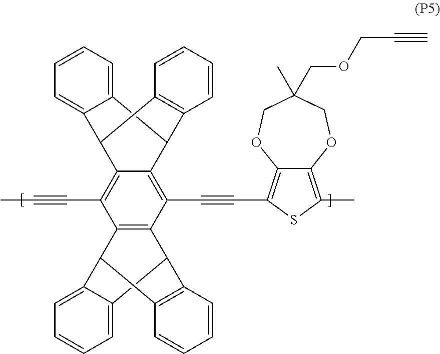

Under an atmosphere of argon, diisopropylamine/toluene (2:3, 5 mL) solvent was added to compound Diethynylpentiptycene (0.04 g, 0.084 mmol) and (6,8-Dibromo-3-methyl-3-prop-2-ynyloxymethyl-3,4-dihydro-2H-thieno[3,4-b][1,4]dioxepine (0.047 g, 0.084 mmol) along with CuI (0.011 g, 0.053 mmol) and Pd(PPh₃)₄ (0.01 g, 8.6 mol). The reaction mixture was then kept for reflux for 5 days at a constant temperature of 65° C. The reaction mixture was then subjected to CHCl₃/H₂O workup. The combined organic layer was then washed with NH₄Cl water solution, and then dried over Na₂SO₄. The solvent was then evaporated to obtain a yellow solid, and was then reprecipitated in methanol thrice. Yield: 81%; ¹H NMR (CDCl₃, 400 MHz, δ ppm): 0.88 (br, 3H), 2.89 (br, 1H), 3.51 (br, 4H), 6.01 (br, 4H), 7.01-7.61 (br, 16H).

The absorption and emission spectra of the polymer was recorded in chloroform. The absorbance spectra shows peak at 376 nm. The emission peak was observed to be at 457 nm. M_{w} of the polymer was found to be approximately 74,000.

### Example 4

### Polymerization of diethynylpentiptycene, 6,8-Dibromo-3,3-Dihexyl-3,4-dihydro-2H-thieno[3,4-b][1,4]dioxepine and 6,8-Dibromo-3,3-Dibenzyl-3,4-dihydro-2H-thieno[3,4-b][1,4]dioxepine (4)

Where x+y=n

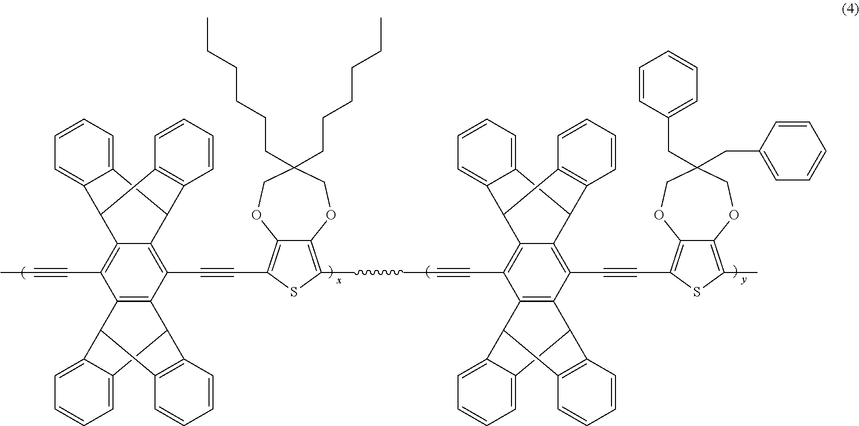

Under an atmosphere of argon, diisopropylamine/toluene (2:3, 5 mL) solvent was added to compound Diethynylpentiptycene (0.04 g, 0.084 mmol) and 6,8-Dibromo-3,3-Dihexyl-3,4-dihydro-2H-thieno[3,4-b][1,4]dioxepine (0.02 g, 0.042 mmol) and 6,8-Dibromo-3,3-Dibenzyl-3,4-dihydro-2H-thieno[3,4-b][1,4]dioxepine (0.021 g, 0.042 mmol) along with CuI (0.011 g, 0.053 mmol) and Pd(PPh₃)₄ (0.01 g, 8.6 mol). The reaction mixture was then kept for reflux for 5 days at a constant temperature of 65° C. The reaction mixture was then subjected to CHCl₃/H₂O workup. The combined organic layer was then washed with NH₄Cl water solution, and then dried over Na₂SO₄. The solvent was then evaporated to obtain a yellow solid, and was then reprecipitated in methanol thrice. Yield: 71%; ¹H NMR (CDCl₃, 400 MHz, δ ppm): 0.86 (b, 6H), 1.2-1.5 (b, 20H), 2.87 (b, 4H), 3.06 (br, 16H), 3.98 (b, 4H), 4.35 (br, 4H), 6.9-7.5 (b, 42H).

The absorption and emission spectra of the polymer was recorded in chloroform. The absorbance spectra shows peak at 411 nm. The emission peak was observed to be at 490 nm. M_{w} of the polymer was found to be approximately 1,230,00.

TABLE 1
Representative structures of polymers:

| | |
|---|---|
| P1 | 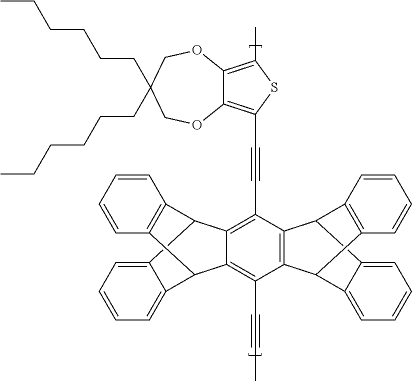 |
| P2 | 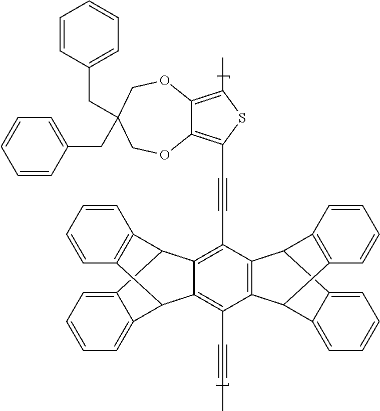 |
| P3 | 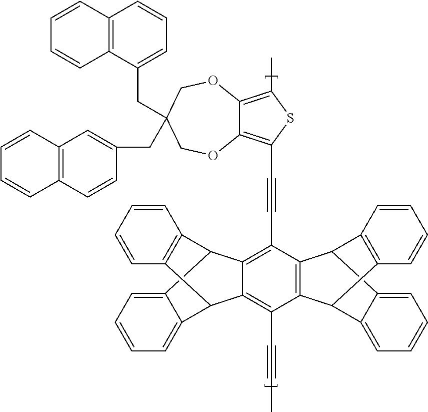 |
| P4 | 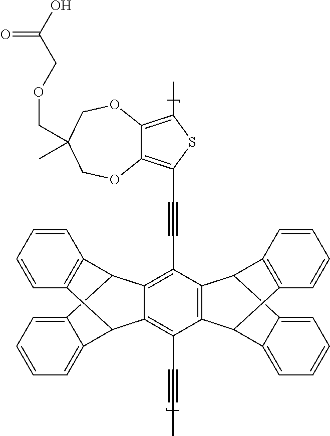 |
| P5 | 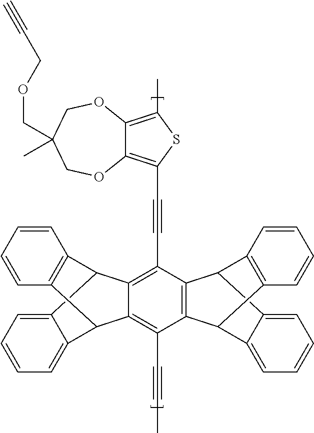 |

### II. Polymers with Acetylenic Functional Group

### A. Quinone

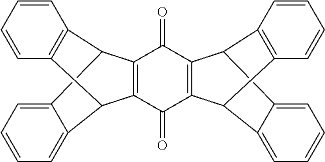

To the mixture of 4 g (22.46 mmol) of anthracene and 1.21 g (11.22 mmol) of benzoquinone was added 50 mL of mesitylene. The mixture was kept for reflux for 24 h and then the solid was filtered after cooling to room temperature. The hydroquinone solid was digested in 100 mL hot xylene twice and filtered. The crude hydroquinone was dissolved in 300 mL of hot glacial acetic acid and then a solution of 0.392 g (2.3 mmol) of potassium bromate in 100 mL of hot water was added. A brown colored precipitate developed immediately. The solution was kept for boiling for few minutes and then additional 100 mL of hot water was added and kept for cooling. The brown colored quinone solid was collected after the solution was cooled. The quinones were washed with acetic acid and then with water. The crude quinones were dissolved in chloroform and washed with sodium bicarbonate and brine. The organic layer was separated and dried over Na₂SO₄. The crude compound was purified using column chromatography, the compound was obtain in 1:1 (ethyl acetate:petroleum ether). The compound remains bound to silica gel which was obtain in chloroform as the eluent. The quinone was obtained as orange solid with chloroform as the eluent. Yield: 23%. ¹H NMR (CDCl₃, δ ppm): 5.75 (s, 4H); 6.97 (dd, 8H, J₁=3.3, J₂=5.3 Hz); 7.36 (dd, 8H, J₁=3.2, J₂=5.3 Hz). ¹³C NMR (CDCl₃): 47.39, 124.24, 125.46, 143.65, 150.95, 179.96. IR (cm⁻¹): 1641, 1471, 1458, 1331, 1319, 1295, 1264, 1202, 1137, 1016, 889, 756, 741. ES-MS: 460 (M⁺). Anal. Calcd for C₃₄H₂₀O₂ (460): C, 88.67; H, 4.38. Found: C, 86.92; H, 3.75.

### B. Trimethylsilyl-protected diethynylpentiptycene

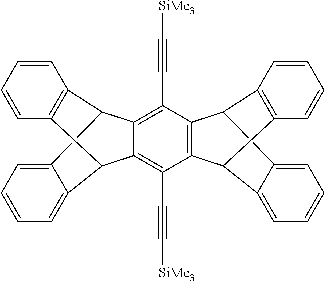

Under an atmosphere of argon, 3.5 mL (2.5 mmol) of n-BuLi in hexane was added dropwise to a solution of 0.76 mL (2.5 mmol) of (trimethylsilyl)acetylene in 1 mL THF at 0° C. The solution was kept for stirring for 40 min at 0° C. and then transferred to the solution of 1 g (2.17 mmol) of quinone in 3 mL THF at 0° C. The solution was then stirred overnight at room temperature. The resulting solution was then quenched with 1 mL of 10% HCl and then extracted from 50 mL of chloroform. The solvent was removed under reduced pressure and hexane was then added to the residue. The resulting pale white solid was collected by filtration. 0.16 g (0.24 mmol) of pale white solid in 10 mL of acetone was added a solution of 0.14 g (2.25 mmol) of tin(II) chloride dihydrated 10 mL of 50% of acetic acid. The mixture was stirred at room temperature for 24 h and then resulting solid product was filtered. To the solid 20 mL of water was added followed by addition of NaHCO₃ and extracted twice from 50 mL of chloroform. The combined organic layer was dried over Na₂SO₄ and concentrated under reduced pressure to yield pale white solid. Yield: 62%. ¹H NMR (CDCl₃, δ ppm): 0.48 (s, 18H), 5.77 (s, 4H), 6.94 (dd, 8H, J₁=3.4, J₂=5.3 Hz), 7.33 (dd, 8H, J₁=3.2, J₂=5.3 Hz). ¹³C NMR (CDCl₃): 0.29, 52.1, 100.5, 102.4, 114.8, 123.7, 125.1, 144.0, 144.8. Anal. Calcd for C₄₄H₃₈Si₂ (622): C, 84.83; H, 6.15. Found: C, 83.49; H, 5.48.

### C. Deprotection of TMS

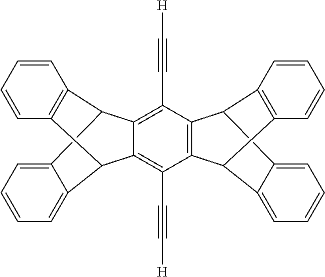

The deprotection of the trimethylsilyl group was carried out by dissolving the TMS protected compound in a mixture of KOH (1 pellet in 1 mL H₂O), THF, and MeOH and stirred at room temperature for 5 h. The resulting white solid was filtered and washed with water and dried. Yield: 95%. ¹H NMR (CDCl₃, δ ppm): 3.69 (s, 2H), 5.82 (s, 4H), 6.94 (dd, 8H, J₁=3.4, J₂=5.3 Hz), 7.33 (dd, 8H, J₁=3.2, J₂=5.3 Hz). ES-MS: 479 (M⁺+1, 32).

### D. 2,5-Dibromo-benzene-1,4-diol

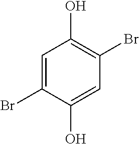

To the solution of 1 g (9.1 mmol) of hydroquinone in 10 mL of acetic acid was added 3.04 g (19.1 mmol) of bromine dropwise at a constant temperature of 10° C. The solution was kept for stirring at 10° C. for 12 h. The resulting solution was then poured in 50 mL of water and extracted from 70 mL of ethyl acetate. The combined organic layer was dried over Na₂SO₄ and concentrated under reduced pressure to yield brown solid. The crude product was purified by silica gel column chromatography eluting with petroleum ether to get white solid. Yield: 77%. ¹H NMR (CDCl₃, δ ppm): 5.2 (s, 2H), 7.1 (s, 2H). ¹³C NMR (CD₃OD): 110.15, 121.11, 149.03. IR (cm⁻¹): 3268, 3094, 2784, 2076, 1703, 1638, 1515, 1429, 1236, 1223, 1200, 1062, 863. MS m/z (relative intensity) 265 (M⁺−2, 51), 267 (M⁺, 100), 269 (M⁺+2, 48). Anal. Calcd for C₆H₆O₂ (267): C, 26.9; H, 1.5. Found: C, 26.7; H, 1.31.

### E. 1,4-Dibromo-2,5-bis-hexyloxy-benzene

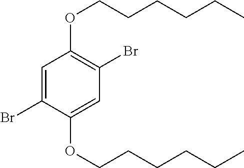

To the solution of 1 g (3.73 mmol) of 2,5-Dibromo-benzene-1,4-diol in 12 mL of DMSO was added 1.13 g (8.21 mmol) of K₂CO₃ under argon atmosphere. The resulting mixture was stirred at for half an hour followed by the addition of 1.35 g (8.21 mmol) of 1-bromohexane dropwise. The mixture was stirred for 48 h under argon atmosphere. The reaction mixture was poured then into 150 mL water and extracted 3-4 times from 80 mL ethyl acetate. The combined organic layer was dried over Na₂SO₄ and concentrated under reduced pressure to get dark brown solid. The crude product was then purified by silica gel column chromatography eluting with petroleum ether, ethyl acetate mixture to get white solid. Yield: 71.2%. ¹H NMR (CDCl₃, δ ppm): 0.92 (t, 6H, J=7.01 Hz), 1.33-1.57 (m, 12H), 1.79 (m, 4H), 3.92 (t, 4H, J=6.41 Hz), 7.17 (s, 2H). ¹³C NMR (CDCl₃): 14.19, 22.75, 25.77, 29.24, 31.65, 70.45, 111.28, 118.61, 150.23. IR (cm⁻¹): 2937, 2853, 1643, 1492, 1461, 1361, 1268, 1211, 1062, 1020, 991, 847, 808, 726. MS m/z (relative intensity) 434 (M⁺−2, 7), 436 (M⁺, 13), 438 (M⁺+2, 6). Anal. Calcd for C₁₈H₂₈Br₂O₂ (436): C, 49.56; H, 6.47. Found: C, 49.22; H, 6.22.

### F. 1,4-Dibromo-2,5-bis-dodecyloxy-benzene

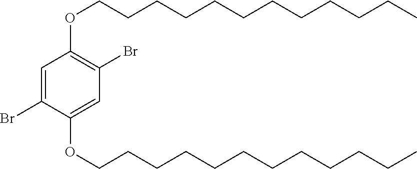

To the solution of 5 g (18.65 mmol) of 2,5-Dibromo-benzene-1,4-diol in 60 mL of DMSO was added 5.66 g (41.03 mmol) of K₂CO₃ under argon atmosphere. The resulting mixture was stirred at for half an hour followed by the addition of 10.22 g (41.03 mmol) of 1-bromododecane dropwise. The mixture was stirred for 48 h under argon atmosphere. The reaction mixture was poured then into 600 mL water and extracted 3-4 times from 60 mL ethyl acetate. The combined organic layer was dried over Na₂SO₄ and concentrated under reduced pressure to get dark brown solid. The crude product was then purified by silica gel column chromatography eluting with petroleum ether, ethyl acetate mixture to get white solid. Yield: 73.6%. ¹H NMR (CDCl₃, δ ppm): 0.88 (t, 6H, J=6.7 Hz), 1.26-1.49 (m, 36H), 1.79 (m, 4H), 3.94 (t, 4H, J=6.41 Hz), 7.08 (s, 2H). ¹³C NMR (CDCl₃): 14.31, 22.88, 26.10, 29.28, 29.47, 29.53, 29.72, 29.76, 29.82, 29.84, 32.10, 70.45, 111.28, 118.59, 150.23. IR (cm⁻¹): 2922, 2850, 1639, 1494, 1459, 1361, 1274, 1213, 1069, 1024, 997, 806. MS m/z (relative intensity) 602 (M⁺−2, 9), 604 (M⁺, 19), 606 (M⁺+2, 10). Anal. Calcd for C₃₀H₅₂Br₂O₂ (604): C, 59.60; H, 8.67. Found: C, 59.11; H, 8.83.

### G. 1,4-Dibromo-2,5-bis-(6-bromo-hexyloxy)-benzene

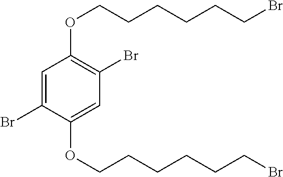

To the solution of 1.5 g (5.59 mmol) of 2,5-Dibromo-benzene-1,4-diol in 20 mL of DMSO was added 1.7 g (12.30 mmol) of K₂CO₃ under argon atmosphere. The resulting mixture was stirred at for half an hour followed by the addition of 13.66 g (55.99 mmol) of 1,6-dibromohexane dropwise. The mixture was stirred for 48 h under argon atmosphere. The reaction mixture was poured then into 100 mL water and extracted 3-4 times from 60 mL ethyl acetate. The combined organic layer was dried over Na₂SO₄ and concentrated under reduced pressure to get dark brown solid. The crude product was then purified by silica gel column chromatography eluting with petroleum ether, ethyl acetate mixture to get white solid. Yield: 77.27%. ¹H NMR (CDCl₃, δ ppm): 1.53 (m, 8H), 1.57-1.92 (m, 8H), 3.43 (t, 4H, J=6.72 Hz), 3.95 (t, 4H, J=6.11 Hz), 7.08 (s, 2H). ¹³C NMR (CDCl₃): 25.37, 27.99, 29.0, 32.82, 33.97, 70.17, 111.32, 118.63, 150.20. IR (cm⁻¹): 2941, 2857, 1776, 1719, 1581, 1492, 1459, 1359, 1298, 1266, 1242, 1211, 1065, 1022, 995, 851, 806, 753, 730, 641. MS m/z (relative intensity) 591 (M⁺−2, 12), 593 (M⁺, 17), 595 (M⁺+2, 11).

### H. 1,4-Dibromo-2,5-diiodobenzene

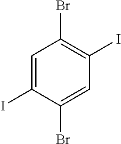

To 2 g (8.4 mmol) of Dibromobenzene was added 25 mL of concentrated H₂SO₄ and reflux till a clear solution is obtained. To the solution, 2.36 g (18.6 mmol) of iodine was added in portions. The resulting purple mixture was stirred at 125-135° C. for 2 days. The reaction mixture was then cooled to room temperature and poured into 100 mL of ice water and extracted with 80 mL of dichloromethane. The dichloromethane layer was then stirred with dilute solution of NaOH in order to remove excess of iodine. The combined organic layer was dried over Na₂SO₄ and concentrated to get yellow solid. Yield: 80%. ¹H NMR (CDCl₃, δ ppm): 8.04 (s).

### I. 4[2,5-Dibromo-4-(3-hydroxy-3-methyl-but-1-ynyl)-phenyl]-2-methyl-but-3-yn-2-ol (Synthesis of Diacetylenic Precursor)

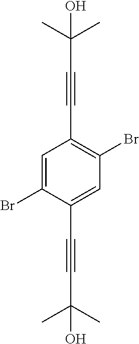

To the solution of 1 g (mmol) of 1,4-Dibromo-2,5-diiodobenzene in 80 mL of benzene and 48 mL of diisopropylamine was added 120 mg (mmol) of CuI followed by the addition of 100 mg (mmol) of Pd(PPh₃)₂Cl₂ under argon atmosphere. To the mixture was then added 1 mL (mmol) of 2-methyl-3-butyn-2-ol and kept to stir at room temperature overnight. The resulting mixture was then poured in 100 mL of water and extracted from 40 mL of ether. The combined organic layer was dried over Na₂SO₄ and concentrated to yield orange solid. Yield: 81%. ¹H NMR (CDCl₃, δ ppm): 1.62 (s, 12H), 2.42 (s, 2H), 7.59 (s, 2H). IR (cm⁻¹): 3271, 2982, 2925, 1507, 1376, 1361, 1276, 1190, 1164, 963.

### Example 5

### General Procedure for Copolymerization Via Coupling of Iptycene and Benzene Derivatives (P6, P7, P8)

Under an atmosphere of argon, diisopropylamine/toluene (2:3, 5 mL) solvent was added to 0.04 g (0.084 mmol) of diacetylene pentiptycene, 0.049 g (0.084 mmol) of 1,4-Dibromo-2,5-bis-(6-bromo-hexyloxy)-benzene/4[2,5-Dibromo-4-(3-hydroxy-3-methyl-but-1-ynyl)-phenyl]-2-methyl-but-3-yn-2-ol/6,8-Dibromo-3-(6-bromo-hexyloxymethyl)-3-methyl-3,4-dihydro-2H thieno[3,4b][1,4]dioxepine followed by the addition of 0.01 g (0.053 mmol) of CuI and 0.009 g (0.008 mmol) of Pd(PPh₃)₄. The mixture was then kept for reflux for 5 days at a constant temperature of 65° C. The resulting mixture was then poured in 100 mL water and extracted twice from 50 mL of chloroform. The chloroform layer was then washed with 5 mL of NH₄Cl water solution. The combined organic layer was dried over Na₂SO₄ and concentrated under reduced pressure to yield brown liquid. The brown liquid was reprecipitated in methanol thrice which resulted in yellow solid.

P6: Yield: 83%. IR (cm⁻¹): 2958, 1552, 1420, 1233, 1010, 760.

P7: Yield: 88%. IR (cm⁻¹): 2946, 2854, 1555, 1448, 1429, 1235, 1056, 759.

P8: Yield: 81%. IR (cm⁻¹): 3203, 2929, 2853, 1503, 1459, 1378, 1261, 1207, 1022, 806, 751.

### Example 6

### General Procedure for the Post Functionalization of P6-P7

To a solution of polymer in THF excess of NaN₃ was added and kept for stirring at room temperature for 12 h. The resulting mixture was then poured in 50 mL water and extracted twice from 75 mL of chloroform. The combined organic layer was dried over Na₂SO₄ and concentrated under reduced pressure to yield yellow liquid The yellow liquid was reprecipitated in methanol thrice which resulted in yellow solid.

P9: Yield: 95%. IR (cm⁻¹): 2963, 2918, 2842, 2056, 1657, 1537, 1370, 1266, 1190, 1090, 1018.

P10: Yield: 92%. IR (cm⁻¹): 2950, 2860, 2153, 2044, 1553, 1507, 1468, 1435, 1255, 1065.

### Example 7

### General Procedure for Deprotection Resulting in Acetylenic Functional Group P11

To a solution of polymer in Toluene excess of NaH was added and kept for reflux for 24 h. The resulting mixture was then poured in 50 mL water and extracted from 70 mL of chloroform. The combined organic layer was dried over Na₂SO₄ and concentrated under reduced pressure to yield yellow liquid. The yellow liquid was reprecipitated in methanol twice which resulted in yellow solid.

P11: Yield: 79%. IR (cm⁻¹): 2962, 2927, 2854, 2386, 1667, 1638, 1391, 1225, 1100, 1044, 973.

### Representations of the Polymers

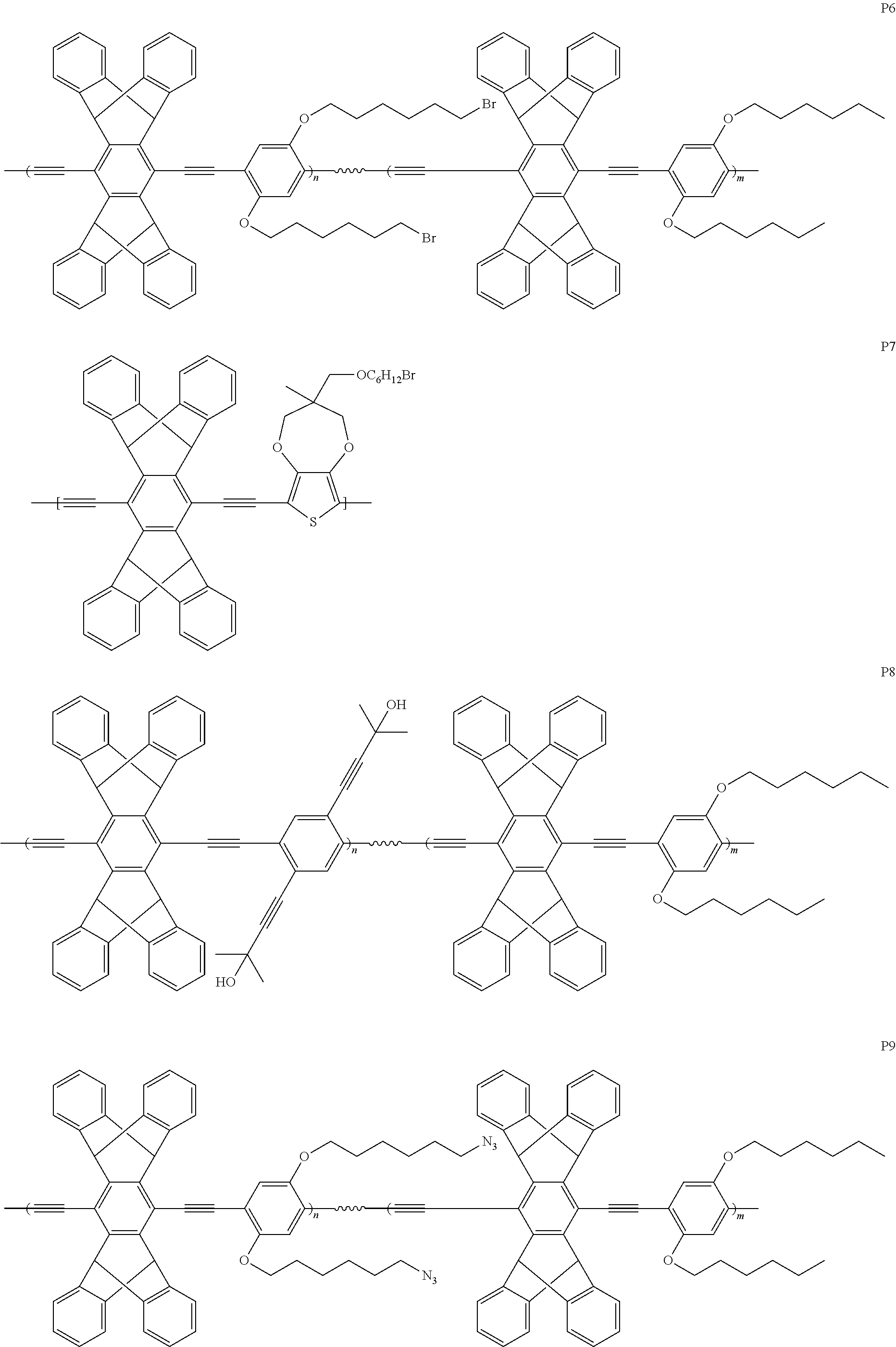

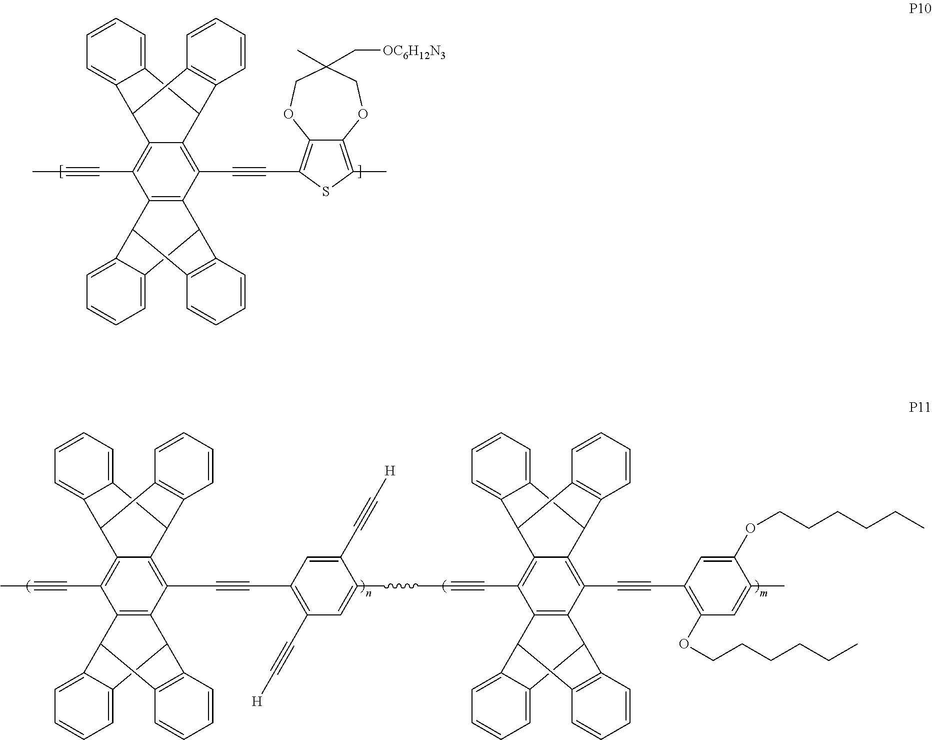

### Example 8

### Synthesis of Clickable Monomers with Acetylenic Functional Group

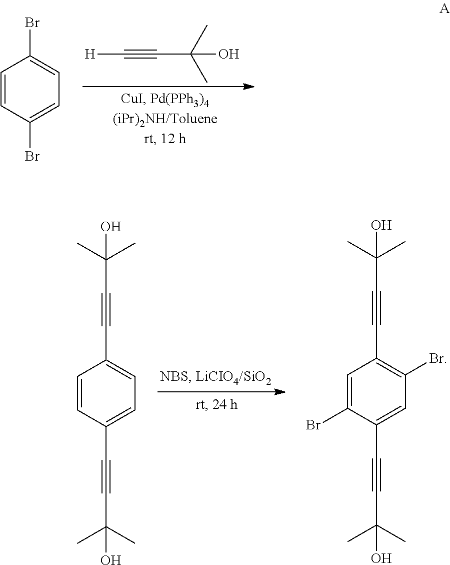

1,4-dibromobenzene was treated with 4-hydroxy-4-methyl butyne in toluene for 12 hours at room temperature in presence of copper iodide, diisopropyl amine and Pd(PPh₃)₄. The resultant product from reaction was brominated using N-bromo succinamide in presence of SiO₂ and LiClO₄ at room temperature for 24 hours.

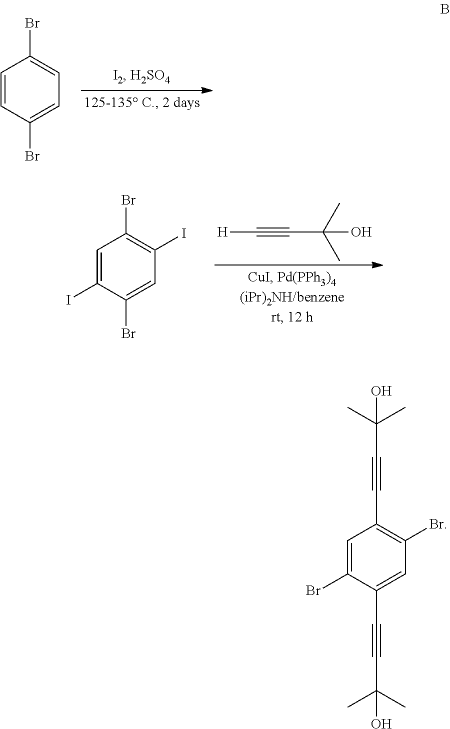

The 1,4-dibromo benzene was treated with Iodine in presence of Conc. H₂SO₄ at 125-135° C. for 2 days and the resultant dibromo-diiodo is treated with 4-hydroxy-4-methyl-butyne for 12 hours in presence of copper iodide, diisopropyl amine and Pd(PPh₃)₄ in benzene. The obtained product is purified.

### C. Synthesis of Clickable Polymer with Acetylenic Functional Group

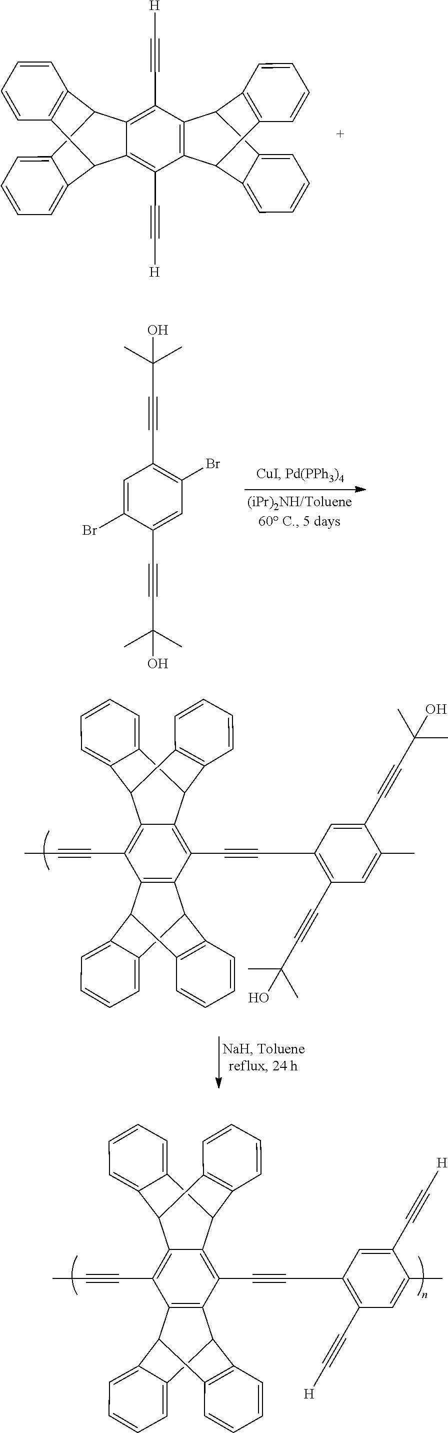

The dibromo compound and the alkyne as shown above are reacted in toluene for 5 days in presence of copper iodide, diisopropyl amine and Pd(PPh₃)₄ at 60° C. The resultant polymer was filtered and refluxed with sodium hydride in toluene for 24 hours under argon atmosphere. The resultant product was filtered and washed with toluene. This is an AFP polymer with an alkyne as pendant group, which will be post derivitized with an azide group containing compound.

### Example 9

### Different Percentage Incorporation of Clickable Unit

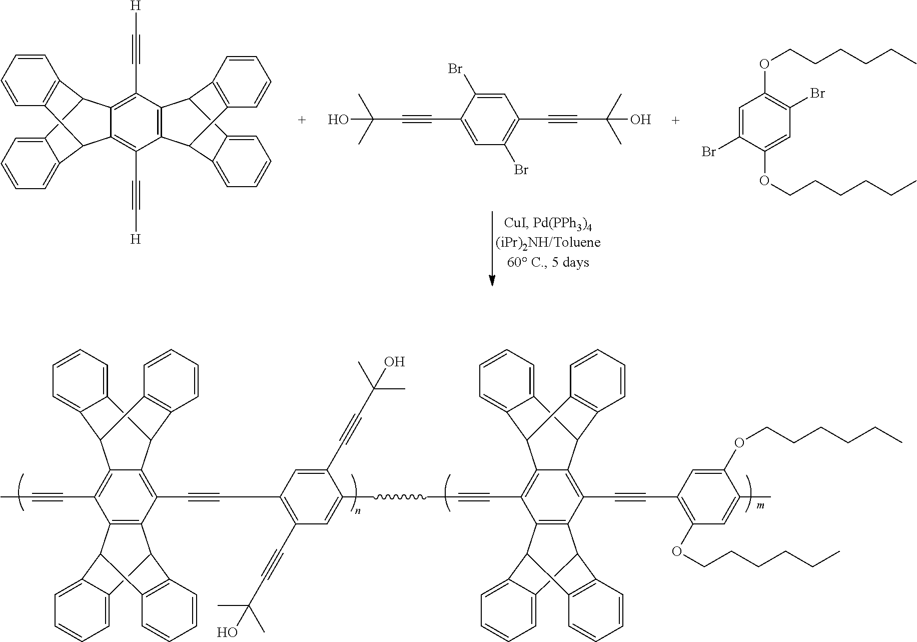

The shown dibromo-dihydroxy and dibromo compounds were treated with alkyne in toluene in presence of copper iodide, Pd(PPh₃)₄, and diisopropylamine at 60° C. for 5 days. Depending on the ratio of dibromo-dihydroxy and dibromo compounds (n:m) used in the reaction, the above AFP polymer is formed and it was filtered and washed with toluene and dried.

### Example 10

### Synthesis of Acetylenic Functionalized Polymer for Click Chemistry

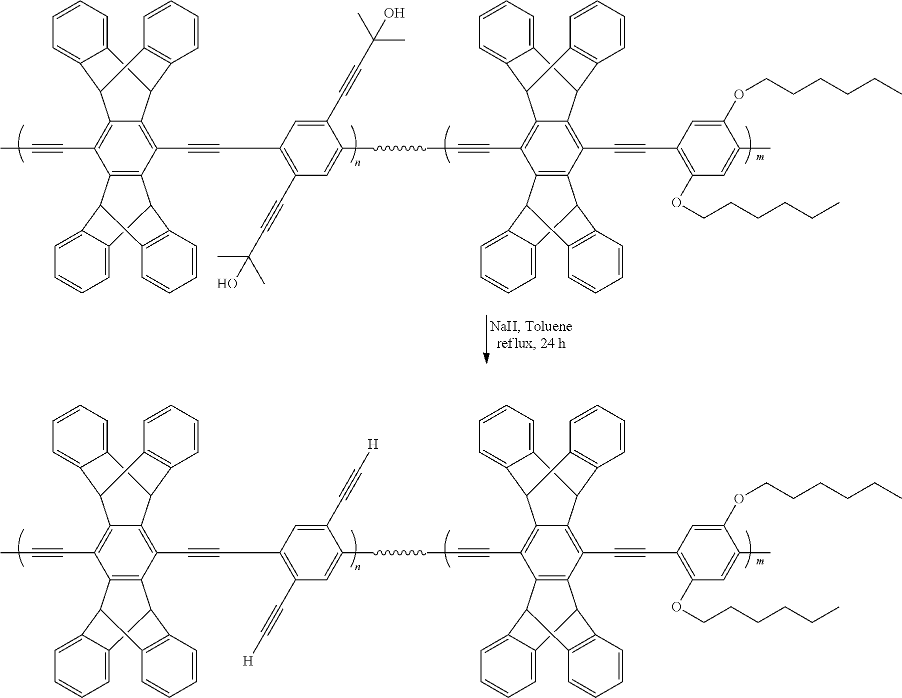

The hydroxy containing polymer was refluxed with sodium hydride in anhydrous toluene under argon atmosphere for 24 hours and the resultant polymer was filtered and washed with hot toluene.

Click chemistry is performed on an alkyne and azide in presence of an organic or aqueous solvent under copper catalyst conditions and the obtained product is called clicked product, which is a triazine derivative.

### III. Preparation of Polymers with Synthesis of Azide Functional Group

### Example 11

### Step 1

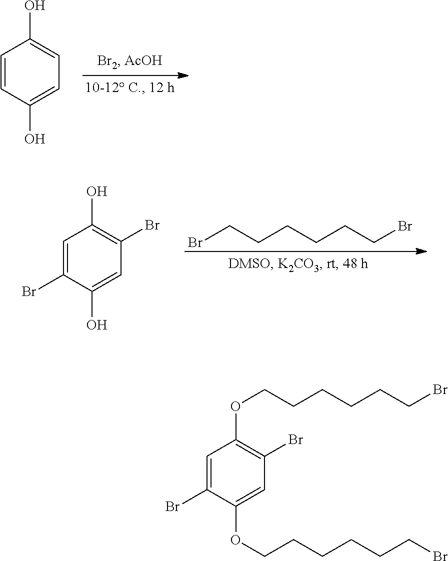

4-hydroxy phenol was treated with bromine in acetic acid at 10-12° C. for 12 hours. Dibromo compound was poured on crushed ice. The precipitated oily solid was extracted in dichloromethane, washed with water, brine, water and organic phase was dried over sodium sulphate and concentrated to yield dibromo compound in 70% yield. The obtained 1,4-dibromo-2-hydroxy phenol was treated with 1,6-dibromohexane in DMSO for 48 hours in presence of K₂CO₃. The reaction mixture was extracted in dichloromethane, and washed with water, sodium bicarbonate, brine and water to obtain an oily compound which was purified by silica gel chromatography.

### Step 2

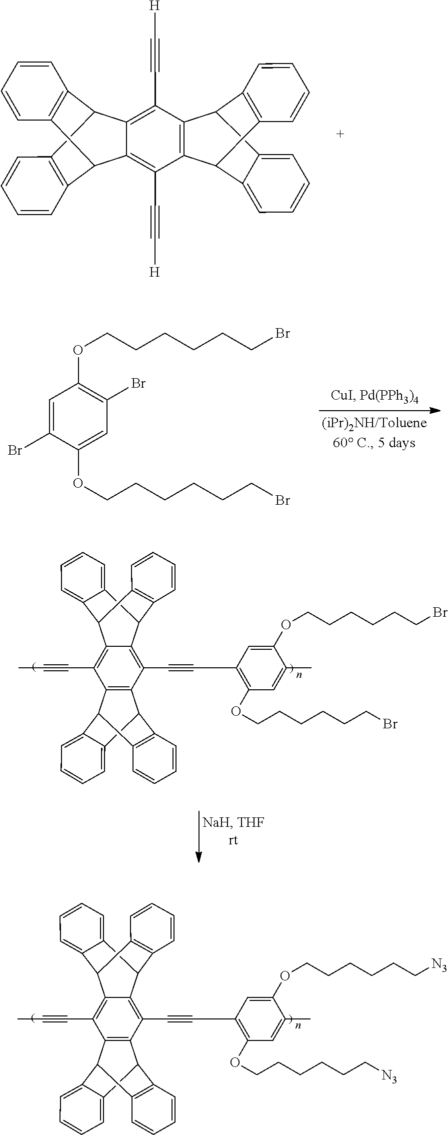

The shown bromo and alkyne were reacted for 5 days at 60° C. in toluene in presence of CuI, Pd(PPh₃)₄ and diisopropylamine. The precipitated polymer was filtered and washed with toluene and air dried. The resultant bromo compound was reacted with sodium azide in THF at room temperature for 12 hours and filtered and washed with THF. Dried compound is an AFP polymer with N₃ as pendant group, which will be post derivitized with an acetylenic group containing compound.

### Example 12

### Different Percentage Incorporation of Clickable Unit

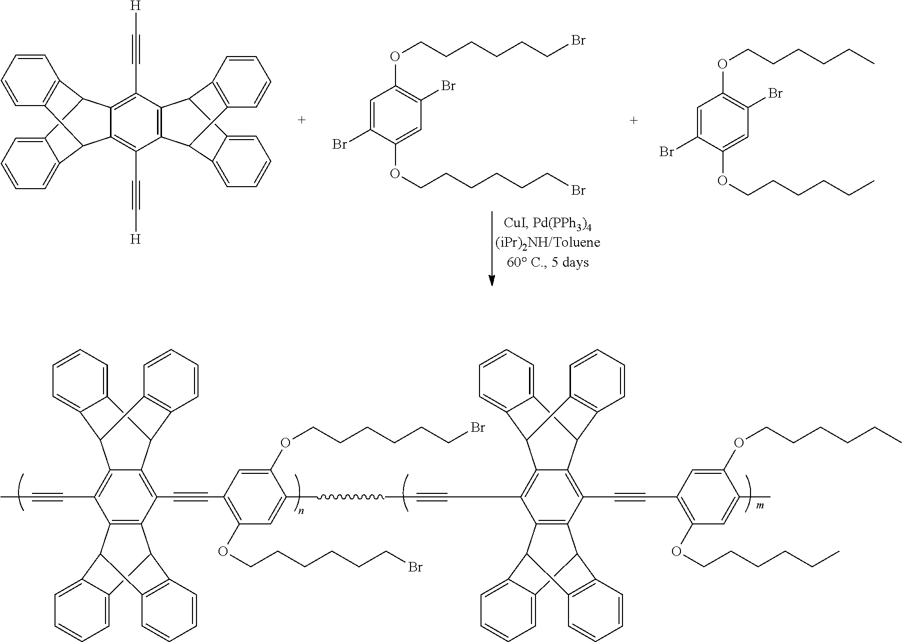

The shown tetra bromo and dibromo compounds were treated with alkyne in toluene in presence of copper iodide, Pd(PPh₃)₄, and diisopropylamine at 60° C. for 5 days. Depending on the ratio of dibromo and tetrabromo compound (m:n) used in the reaction, the above AFP polymer is formed and it was filtered and washed with toluene and dried.

### Example 13

### Synthesis of Azide Functionalized Polymer for Click Chemistry

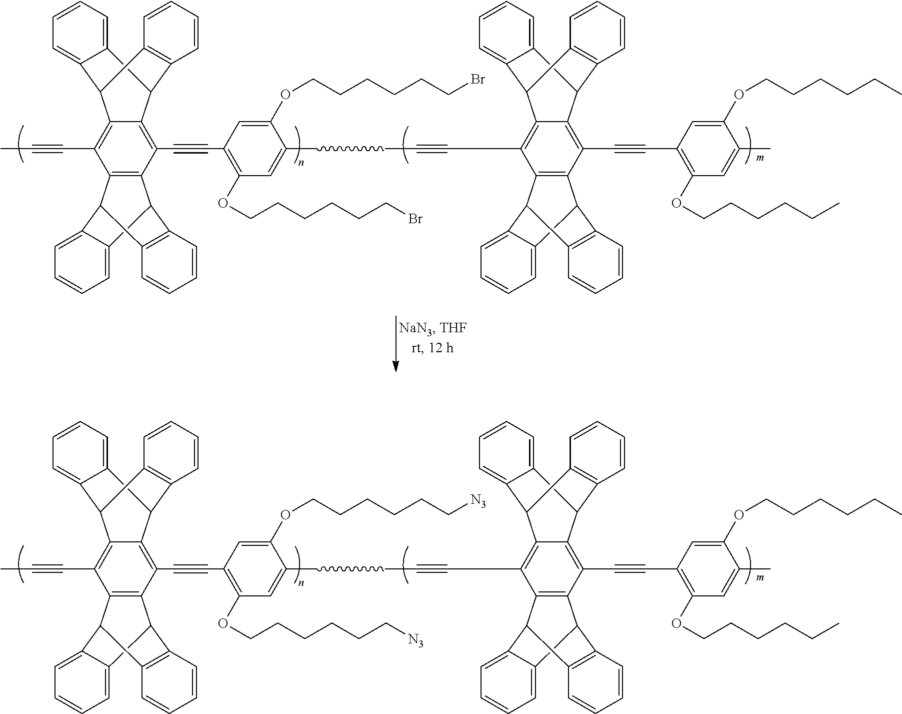

The dibromo containing AFP polymer was treated with sodium azide in THF for 12 hours at room temperature and the obtained pendant group containing AFP polymer was filtered and washed with THF and dried. This is an AFP polymer with azide group as pendant, which will be post derivitized with an acetylenic group containing compound.

The present disclosure hence provides compounds which can be used in the detection of various analytes.

1. A compound of formula I, 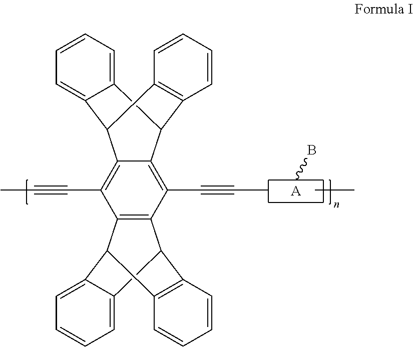
Wherein
B is selected from a group comprising —O—R—CCH, —O—CCH and —O—R—N₃;
R is selected from a group comprising but not limiting to linear or branched aliphatic C₁-C₂₀ alkyl chain, cycloaliphatic C₃-C₂₀ compound and aromatic C₅-C₂₀ compound optionally substituted with suitable functional group selected from a group comprising OH, N₃, R₁CCH, NH₂, NO₂, CHO, COOH and CN;
A is selected from a group comprising aryl, heteroaryl, cycloalkyl, heterocycloalkyl groups optionally substituted with suitable functional groups selected from a group comprising OH, N₃, R₁CCH, NH₂, NO₂, CHO, COOH and CN;
R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and
n ranges from 1 to about 15,000.

2. A compound of formula II, 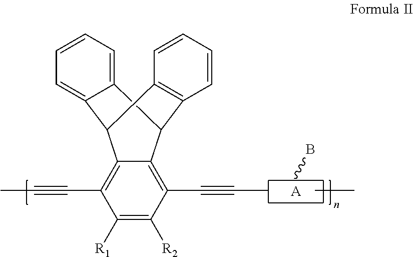
Wherein
B is selected from a group comprising —O—R—CCH, —O—CCH and —O—R—N₃;
R is selected from a group comprising but not limiting to linear or branched aliphatic C₁-C₂₀ alkyl chain, cycloaliphatic C₃-C₂₀ compound and aromatic C₅-C₂₀ compound; optionally substituted with suitable functional group selected from a group comprising OH, N₃, R₁CCH, NH₂, NO₂, CHO, COOH and CN;
A is selected from a group comprising aryl, heteroaryl, cycloalkyl, heterocycloalkyl groups; optionally substituted with suitable functional groups selected from a group comprising OH, N₃, R₁CCH, NH₂, NO₂, CHO, COOH and CN;
R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN; and
n ranges from 1 to about 15,000.

3. The compound as claimed in claims 1 and 2, wherein A is selected from a group comprising 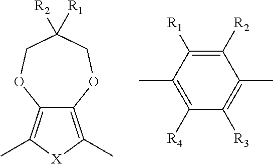
wherein
R₁ is selected from a group comprising but not limiting to the B, linear or branched aliphatic C₁-C₂₀ alkyl chain, cycloaliphatic C₃-C₂₀ compound and aromatic C₅-C₂₀ compound comprising the B; optionally substituted with functional groups comprising OH, N₃, R₁CCH, NH₂, NO₂, CHO, COOH and CN;
X is selected from a group comprising but not limiting to O, S and NR;
R₂ is selected from a group comprising but not limiting to the B, linear or branched aliphatic C₁-C₂₀ alkyl chain, cycloaliphatic C₃-C₂₀ compound, and aromatic C₅-C₂₀ compound comprising B; optionally substituted with functional groups comprising OH, N₃, R₁CCH, NH₂, NO₂, CHO, COOH and CN;
R₃ is selected from a group comprising but not limiting to the B, linear or branched aliphatic C₁-C₂₀ alkyl chain, cycloaliphatic C₃-C₂₀, and aromatic C₅-C₂₀ compound comprising the B; optionally substituted with functional groups comprising OH, N₃, R₁CCH, NH₂, NO₂, CHO, COOH and CN; and
R₄ is selected from a group comprising but not limiting to the B, linear or branched, aliphatic C₁-C₂₀ alkyl chain, cycloaliphatic C₃-C₂₀ compound, aromatic C₅-C₂₀ compound comprising the B; optionally substituted with functional groups comprising OH, N₃, R₁CCH, NH₂, NO₂, CHO, COOH and CN.

4. A compound of formula III,
—P-Q- Formula III
Wherein,
P is amplified fluorescent polymer of formula I of claim 1 or formula II of claim 2; and
Q is selected from a group comprising

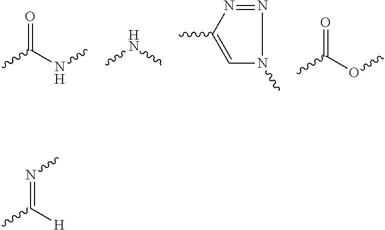

5. A sensor for detecting an analyte comprising compound of formula III;
—P-Q- Formula III
P is amplified fluorescent polymer of formula I of claim 1 or formula II of claim 2; and
Q is selected from a group comprising

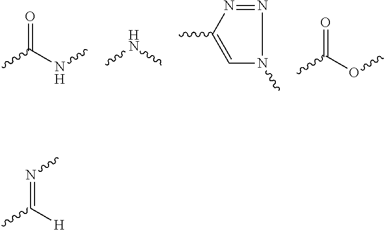

6. The sensor as claimed in claim 5, wherein the sensor comprises buffer, alcohol and detector.

7. The sensor as claimed in claim 6, wherein the buffer is selected from a group comprising sodium acetate, potassium acetate, piperidine, ethanolamine, pyridine, pyrizine, tristriphine, MOPS and MES.

8. The sensor as claimed in claim 6, wherein the alcohol is selected but not limiting to a group comprising methyl alcohol, ethyl alcohol, isopropyl alcohol, n-butyl alcohol and t-butyl alcohol.

9. The sensor as claimed in claim 6, wherein the detector is fluorescence detector.

10. A method of detection of an analyte in a sample, said method comprising steps of;
a. contacting compound of formula III or sample comprising compound of formula III with sample in presence of a solvent; and
b. determining change in fluorescence of the reacted compound of formula III to detect the analyte.

11. The method of detection as claimed in claim 10, wherein concentration of the analyte is detected.

12. The method of detection as claimed in claim 10, wherein the detection of the analyte is with the help of sensor claimed in claim 5.

13. The method of detection as claimed in claim 10, wherein the solvent is selected from a group not limiting to dichloromethane, chloroform, water, aqueous buffer with pH ranging from about 2 to about 12, preferably ranging from about 5 to about 9, hexane, ethyl acetate, acetone, toluene, xylene, dimethyl sulphoxide, N,N-dimethyl formamide, acetic acid, formic acid, H₂SO₄, HCl and HNO₃

14. The method of detection as claimed in claim 10, wherein the analyte is a solid, liquid, gas and mixture thereof.

15. The method of detection as claimed in claim 10, wherein the analyte is selected from a group comprising chemical analyte and biochemical analyte.

16. The method of detection as claimed in claim 15, wherein the chemical analyte is selected from a group comprising but not limiting to samples obtained from environmental studies and industrial effluents.

17. The method of detection as claimed in claim 16, wherein the sample is selected from a group comprising organo phosphates, nitro compounds, electrophiles and nucloephiles.

18. The method of detection as claimed in claim 15, wherein the biochemical analyte is extracted from a biological sample selected from a group comprising serum, blood, plasma, saliva, urine, feces, seminal plasma, sweat, liquor, amniotic fluid, tissue homogenate and ascites.

19. The method as claimed in claim 18, wherein the biochemical analyte is selected from a group comprising but not limiting to peptides, proteins, antibodies, hormone, lecithin, enzymes, DNA and RNA.
